(19) United States
(12) Patent Application Publication
(10) Pub. No.: US 2010/0048918 A1
(43) Pub. Date: Feb. 25, 2010

(54) Method of manufacturing vinylethylene carbonate

(75) Inventors: In-Soo Yeo, Ulsan-city (KR); Byung-Won Woo, Ulsan-city (KR); Seoung-Woo Yoon, Ulsan-city (KR); Joo-Ho Lee, Ulsan-city (KR); Soon-Hong Park, Busan-city (KR); Nak-Jook Jang, Ulsan-city (KR)

Correspondence Address: HARNESS, DICKEY & PIERCE, P.L.C., P.O. BOX 8910, RESTON, VA, 20195, US

(73) Assignee: FOOSUNG CO., LTD.

(21) Appl. No.: 12/320,094

(22) Filed: Jan. 16, 2009

(30) Foreign Application Priority Data
Aug. 20, 2008 (KR) 10-2008-0081419

(51) Int. Cl. C07D 317/26

(52) U.S. Cl. 549/230

(57) ABSTRACT

Provided is a method of manufacturing vinylethylene carbonate. The method includes synthesizing vinylethylene carbonate (VEC) by reaction of 3,4-dihydroxy-1-butene (3,4-DHB) and dialkyl carbonate using a base catalyst, and refining the VEC.

### CROSS-REFERENCE TO RELATED APPLICATION

This application claims the benefit of Korean Patent Application No. 2008-0081419, filed Aug. 20, 2008, the disclosure of which is hereby incorporated herein by reference in its entirety.

### BACKGROUND OF THE INVENTION

1. Field of the Invention

The present invention relates to a method of manufacturing cyclic carbonate, and more particularly, to a method of manufacturing vinylethylene carbonate.

2. Description of the Related Art

Lithium secondary batteries generally use a non-aqueous organic electrolytic solution in which LiPF₆ is dissolved in an organic solvent. The electrolytic solution should have high conductivity, high electrical, chemical and thermal stabilities, and low reactivity to a container and electrode materials, particularly oxidative stability with respect to a cathode material and reductive stability with respect to an anode material. However, there is no organic solvent known to have all of these characteristics, and thus a mixed organic solvent is used. In an actual battery manufacturing process, additives, e.g., vinylenecarbonate (VC), fluoroethylenecarbonate (FEC), vinylethylenecarbonate (VEC) and propane sulton (PS), are used to improve battery performance.

Among these additives, VEC may be manufactured using butadiene monoxide (BMO) as a reactant. However, BMO is difficult to transport and store because it is explosive, and thus is rarely produced commercially. In addition, in order to manufacture VEC using BMO as a reactant, a reaction has to be performed using a costly metal catalyst under high temperature and pressure, and the yield of VEC is very low.

### SUMMARY OF THE INVENTION

The present invention provide a method of manufacturing a vinylethylene carbonate (VEC) by which superior conversion efficiency to VEC at low temperature, and high-purity VEC can be obtained.

According to an aspect of the present invention, a method of manufacturing VEC is provided. The method includes synthesizing VEC by reaction of 3,4-dihydroxy-1-butene (3,4-DHB) and dialkyl carbonate using a base catalyst.

According to another aspect of the present invention, a method of manufacturing vinylethylene carbonate is provided. The method includes putting 3,4-dihydroxy-1-butene (3,4-DHB), dialkyl carbonate and a base catalyst into a reactor. A product mixture containing vinylethylene carbonate (VEC) and alcohol is obtained by reaction of 3,4-DHB and dialkyl carbonate. Alcohol is removed from the obtained product mixture. The alcohol-free product mixture is distilled, thereby obtaining purified vinylethylene carbonate.

### BRIEF DESCRIPTION OF THE DRAWINGS

These and other aspects and advantages of the invention will become apparent and more readily appreciated from the following description of example embodiments taken in conjunction with the accompanying drawings, of which:

FIG. 1 is a flowchart of a method of manufacturing vinylethylene carbonate according to an example embodiment of the present invention; and

FIG. 2 is a schematic diagram of equipment for manufacturing vinylethylene carbonate according to an example embodiment of the present invention.

### DETAILED DESCRIPTION OF THE INVENTION

Reference will now be made in detail to example embodiments of the present invention shown in the accompanying drawings.

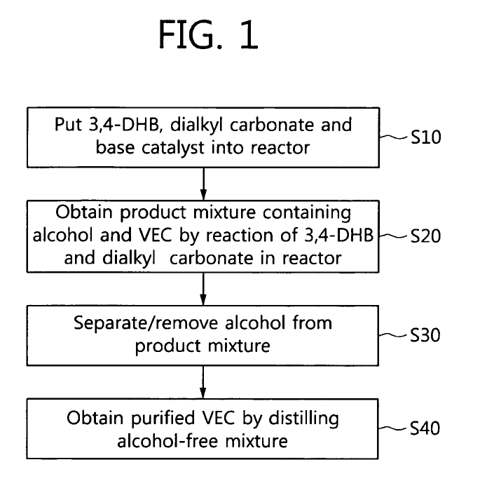

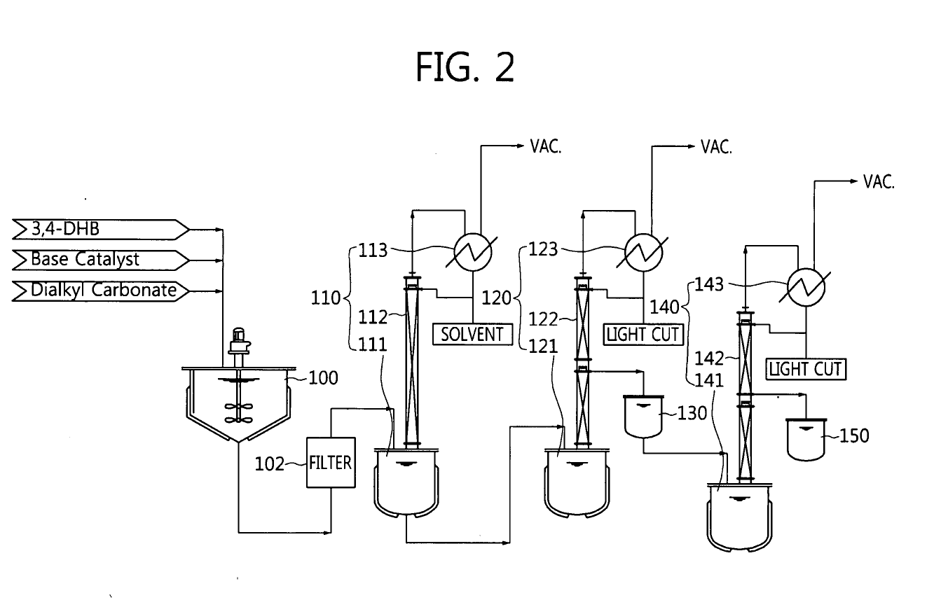

FIG. 1 is a flowchart of a method of manufacturing vinylethylene carbonate (VEC) according to an example embodiment of the present invention. FIG. 2 is a schematic diagram of equipment for manufacturing VEC.

Referring to FIGS. 1 and 2, reactants, e.g., 3,4-dihydroxy-1-butene (3,4-DHB) and dialkyl carbonate and a base catalyst are put into a reactor 100 (S10). As illustrated in Reaction Formula 1,3,4-DHB is reacted with dialkyl carbonate using the base catalyst in the reactor 100 to yield a product mixture containing alcohol and VEC (S20).

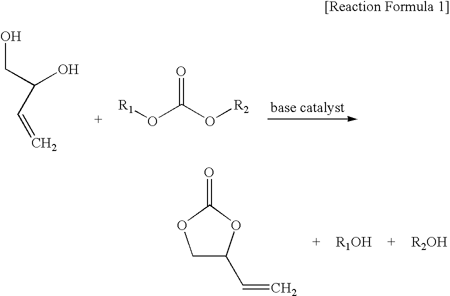

In Reaction Formula 1, R₁ and R₂ are alkyl groups having 1 to 3 carbon atoms that are not related to each other.

The dialkyl carbonate may be dimethyl carbonate (DMC). In this case, the alcohol produced in Reaction Formula 1 may be methanol. The alcohol produced in Reaction Formula 1 may serve to increase activity of the base catalyst.

The base catalyst may be M(ORₓ,). Here, M may be an alkali metal or alkali earth metal, and Rx may be an alkyl group having 1 to 3 carbon atoms. For example, the base catalyst may be sodium methoxide. The base catalyst may be provided by being added to a solvent, specifically alcohol. For example, the base catalyst may be SM-30, a solution containing 30 wt % sodium methoxide in methanol.

For every 1 mole of 3,4-DHB, 1.5 to 2.5 moles, and preferably about 2 moles of the dialkyl carbonate may be added. In addition, considering the aim of high VEC production and low production of a high boiling point material, 1.5 to 3.1 parts by weight of the base catalyst may be added per 100 parts by weight of 3,4-DHB. When the base catalyst is sodium methoxide, 5 to 10 parts by weight of SM-30, the solution containing 30 wt % sodium methoxide in methanol, may be added per 100 parts by weight of 3,4-DHB.

The base catalyst M(ORₓ) may react with moisture, thereby producing MOH. In this case, butadiene monoxide (BMO), which is a low boiling point material, may be produced in the reactor, and a side reaction of the MOH with 3,4-DHB to produce an —OM type salt may occur. Also, the MOH may re-decompose the VEC produced during the reaction and produce a high boiling point material. Thus, before the reactants such as 3,4-DHB and dialkyl carbonate are put into the reactor, moisture may be removed.

The reactor 100 may be a batch-type reactor which simultaneously induces the reactants and the catalyst to react. The batch-type reactor may further increase conversion efficiency of the reaction described as the reaction formula 1. Alternatively, the reactor may be a continuous- or semibatch-type reactor.

During the reaction in the reactor 100, a refluxed material may be generally the alcohol produced by Reaction Formula 1. Particularly, since methanol is flammable, it has to be refluxed while vapor is sufficiently condensed not to leak out, in order to prevent a fire.

The reaction temperature in the reactor 100 may be 30 to 100° C. and the reaction gauge pressure may be 0 to 0.2 kgf/cm²G Accordingly, the side reaction producing BMO may be prevented by low temperature reaction under a substantial atmospheric pressure. Such a reaction may be performed for 30 minutes to 4 hours. Preferably, for higher VEC production, the reaction may be performed for 30 minutes to 2 hours, and more preferably 30 minutes to 1 hour.

Afterward, the product mixture may be filtered through a filter 102. The filtered product mixture is put into a solvent remover 110 to remove alcohol from the product mixture (S30). Here, unreacted dialkyl carbonate may also be removed. Particularly, when the solvent is methanol, it may be removed at 50 to 65° C., and when the solvent is dimethyl carbonate, it may be removed at 85 to 90° C. The solvent remover 110 may be operated at sub-atmospheric pressure.

The solvent remover 110 may be a rotatory evaporator or a distillation tower for removing solvent. When the solvent remover 110 is a distillation tower for removing solvent, it may include a reboiler 111, a distillation column 112 installed on the reboiler 111, and a condenser 113 connected to a top portion of the distillation column 112. In order to operate the solvent remover 110 at sub-atmospheric pressure, the solvent remover 110 may be further connected to a vacuum pump. The vacuum pump may be a dry vacuum pump that does not use oil.

The alcohol-free mixture is distilled to obtain purified VEC (S40). Particularly, a first purified material, crude VEC, may be obtained from the alcohol-free mixture by first distillation, and purified VEC may be obtained from the crude VEC by second distillation. The first and second distillation steps may be performed at sub-atmospheric pressure. Distillation at sub-atmospheric pressure may decrease distillation temperature, and thus a side reaction and production of a high boiling point material may be prevented.

In the first distillation, the alcohol-free mixture is distilled in a first distiller 120 to obtain crude VEC. The first distiller 120 may include a reboiler 121, a distillation column 122 installed on the reboiler 121, and a condenser 123 connected to a top portion of the distillation column 122. Particularly, when the alcohol-free mixture is put into the reboiler 121, the reboiler 121 may be operated at 100 to 110° C., the distillation column 122 may be operated at 0.1 to 5 torr, and the condenser 123 may be operated at 30° C. or less. Here, unreacted reactants containing low boiling point dialkyl carbonate and 3,4-DHB, alcohol and moisture may be extracted and removed from the top portion of the first distillation column 122, high boiling point materials may be extracted and removed from a bottom portion of the first distillation column 122, and a first purified material, crude VEC, may be obtained from a side-cut of the first distillation column 122. The obtained crude VEC may be stored in a first container 130. The first distiller 120 may be a batch- or continuous-type distiller.

Before the first distillation, the distillation column 122 may be operated at a temperature lower than that for the first distillation, to more effectively evaporate and remove the low boiling point materials, i.e., the unreacted reactants, alcohol and moisture from the mixture. To be specific, the distillation column 122 may be operated under a pressure of 1.0 torr or less and at a reboiler temperature of 95 to 110° C.

The moisture content in the crude VEC stored in the first container 130 is analyzed. When at least 100 ppm moisture is contained, the crude VEC may be filtered through a moisture removing process using an absorbent, e.g., a molecular sieve, and then put into a second distiller 140. The molecular sieve may be a molecular sieve 4A having a pore size of 4 Å. In some cases, the first distiller 120 may be reused without separately equipping the second distiller 140.

In the second distillation, the crude VEC is secondarily distilled in the second distiller 140, thereby obtaining purified VEC. The second distiller 140 may include a reboiler 141, a distillation column 142 installed on the reboiler 141, and a condenser 143 connected to a top portion of the distillation column 142. To be specific, the crude VEC is put into the reboiler 141, which may be operated at 115 to 120° C., the distillation column 142 may be operated at 0.5 to 1.0 torr, and the condenser 143 may be operated at 30° C. or less. As a result, unreacted reactants containing low boiling point materials such as dialkyl carbonate and 3,4-DHB, alcohol and moisture may be removed again from a top portion of the distillation column 142 that is a reflux line, high boiling point materials may be removed again from the bottom portion of the distillation column 142 that is the reboiler 141, and thus purified VEC may be obtained from a side-cut of the distillation column 142. The obtained purified VEC may be stored in a second container 150.

Before the second distillation, the distillation column 142 may first be operated at a lower temperature than in the second distillation to evaporate and remove low boiling point materials, such as unreacted reactant, alcohol and moisture from the crude VEC mixture. To be specific, while the distillation column 142 is operated at 0.5 to 1.0 torr, the reboiler 141 is operated at a temperature of 95 to 100° C. and the condenser 143 is operated at a temperature of 30° C. or less, a refluxed solution may be obtained from a reflux line of the second distillation column, and low boiling point materials may be removed therefrom. In this process, a refluxed solution is obtained from a side-cut in the middle of the distillation column to analyze the purity of the VEC. When the purity of VEC reaches an appropriate level, the temperature of the reboiler 141 increases to 115 to 120° C., and thus a second purified product containing at least 99.9 wt % VEC, purified VEC, may be obtained.

As described above, a product mixture containing VEC may be manufactured by a single process performed in the reactor. To be specific, 3,4-DHB and dialkyl carbonate may be reacted together in the presence of a base catalyst, thereby synthesizing VEC without an additional process. Also, the product mixture including VEC may be distilled at sub-atmospheric pressure, and simply separated and purified, thereby obtaining high-purity VEC. The above-described method is simpler and less dangerous than other methods of manufacturing VEC and enables high-purity VEC to be manufactured economically.

Hereinafter, examples of the invention will be described in detail to flesh out the present disclosure. However, the following examples are provided only to aid in understanding the present invention, not to limit its scope.

<Example of Synthesizing VEC 1>

4050 g of 3,4-DHB (45.96 mol), 8280 g of DMC (91.93 mol) and 412 g of SM-30 (parts by weight of SM per 100 parts by weight of 3,4-DHB: 3.1) were put into a batch-type reactor, and reaction was performed for a total of 2 hours at a temperature of 30° C. and a pressure of 0.2 kgf/cm²G or less. The compositions of product mixtures after 1- and 2-hour reactions are shown in Table 1.

<Example of Synthesizing VEC 2>

4100 g of 3,4-DHB (46.53 mol), 8380 g of DMC (93.03 mol) and 417 g of SM-30 (parts by weight of SM per 100 parts by weight of 3,4-DHB: 3.1) were put into a batch-type reactor, and reaction was performed for a total of 2 hours at a temperature of 75° C. and a pressure of 0.2 kgf/cm²G or less. The compositions of product mixtures after 1- and 2-hour reactions are shown in Table 1.

<Example of Synthesizing VEC 3>

4080 g of 3,4-DHB (46.30 mol), 8340 g of DMC (92.58 mol) and 415 g of SM-30 (parts by weight of SM per 100 parts by weight of 3,4-DHB: 3.1) were put into a batch-type reactor, and reaction was performed for a total of 2 hours at a temperature of 95° C. and a pressure of 0.2 kgf/cm²G or less. The compositions of product mixtures after 1- and 2-hour reactions are shown in Table 1.

<Example of Synthesizing VEC 4>

5000 g of 3,4-DHB (56.75 mol), 10220 g of DMC (113.45 mol) and 258 g of SM-30 (parts by weight of SM per 100 parts by weight of 3,4-DHB: 1.5) were put into a batch-type reactor, and reaction was performed for a total of 2 hours at a temperature of 30° C. and a pressure of 0.2 kgf/cm²G or less. The compositions of product mixtures after 1- and 2-hour reactions are shown in Table 1.

<Example of Synthesizing VEC 5>

5000 g of 3,4-DHB (56.75 mol), 10220 g of DMC (113.45 mol) and 258 g of SM-30 (parts by weight of SM per 100 parts by weight of 3,4-DHB: 1.5) were put into a batch-type reactor, and reaction was performed for a total of 2 hours at a temperature of 75° C. and a pressure of 0.2 kgf/cm²G or less. The compositions of product mixtures after 1- and 2-hour reactions are shown in Table 1.

<Example of Synthesizing VEC 6>

5000 g of 3,4-DHB (56.75 mol), 10220 g of DMC (113.45 mol) and 258 g of SM-30 (parts by weight of SM per 100 parts by weight of 3,4-DHB: 1.5) were put into a batch-type reactor, and the reaction was performed for a total of 2 hours at a temperature of 95° C. and a pressure of 0.2 kgf/cm²G or less. The compositions of product mixtures after 1- and 2-hour reactions are shown in Table 1.

<Example of Synthesizing VEC 7>

6000 g of 3,4-DHB (68.1 mol), 12500 g of DMC (138.8 mol) and 610 g of SM-30 (parts by weight of SM per 100 parts by weight of 3,4-DHB: 3.1) were put into a batch-type reactor, and reaction was performed for a total of 2 hours at a temperature of 30° C. and a pressure of 0.2 kgf/cm²G or less. The compositions of product mixtures after 1- and 2-hour reactions are shown in Table 1.

<Example of Synthesizing VEC 8>

6000 g of 3,4-DHB (68.1 mol), 12500 g of DMC (138.8 mol) and 305 g of SM-30 (parts by weight of SM per 100 parts by weight of 3,4-DHB: 1.5) were put into a batch-type reactor, and reaction was performed for a total of 2 hours at a temperature of 30° C. and a pressure of 0.2 kgf/cm²G or less. The compositions of product mixtures after 1- and 2-hour reactions are shown in Table 1.

<Example of Synthesizing VEC 9>

1000 g of 3,4-DHB (11.35 mol), 2040 g of DMC (22.65 mol) and 103 g of SM-30 (parts by weight of SM per 100 parts by weight of 3,4-DHB: 3.1) were put into a batch-type reactor, and reaction was performed for a total of 24 hours at a temperature of 30° C. and a pressure of 0.2 kgf/cm²G or less. The compositions of product mixtures after 1-, 2- and 24-hour reactions are shown in Table 1.

<Example of Synthesizing VEC 10>

1015 g of 3,4-DHB (11.52 mol), 2070 g of DMC (22.98 mol) and 27 g of SM-30 (parts by weight of SM per 100 parts by weight of 3,4-DHB: 0.8) were put into a batch-type reactor, and reaction was performed for a total of 2 hours at a temperature of 30° C. and a pressure of 0.2 kgf/cm²G or less. The compositions of product mixtures after 1- and 2-hour reactions are shown in Table 1.

<Example of Synthesizing VEC 11>

1008 g of 3,4-DHB (11.44 mol), 2060 g of DMC (22.87 mol) and 151 g of SM-30 (parts by weight of SM per 100 parts by weight of 3,4-DHB: 4.5) were put into a batch-type reactor, and reaction was performed for a total of 2 hours at a temperature of 30° C. and a pressure of 0.2 kgf/cm²G or less. The compositions of product mixtures after 1- and 2-hour reactions are shown in Table 1.

<Comparative Example of Synthesizing VEC>

500 g of 3,4-DHB (5.67 mol) and 1020 g of DMC (10.29 mol) were put into a batch-type reactor, and reaction was performed for a total of 24 hours at a temperature of 30° C. and a pressure of 0.2 kgf/cm²G or less in the absence of SM-30 catalyst. The compositions of product mixtures after 4- and 24-hour reactions are shown in Table 1.

The ingredients of the product mixtures obtained from Examples 1 to 11 and Comparative example were analyzed, and the results are shown in Table 1.

TABLE 1

| | | | | | | | Product mixture | | | |
|---|---|---|---|---|---|---|---|---|---|---|
| | Reaction mixture (g) | | | | | | (after removal of DMC) | | | |
| | 3,4-DHB | DMC | SM30 | Reaction conditions | | | | Unreacted | | |
| | (g) | (g) | (g) | Temp. | Pressure | Time | VEC | 3,4-DHB | BMO | Others |
| | ((mol)) | ((mol)) | (parts by weight⁽¹⁾) | (° C.) | (kgf/cm²G) | (hr) | (wt %) | (wt %) | (wt %) | (wt %) |
| Example 1 | 4050 | 8280 | 412 | 30 | 0.2 [symbol] | 1.0 | 92.28 | 6.11 | 0.43 | 1.18 |
| | (45.96) | (91.93) | 3.1 | | | 2.0 | 89.23 | 9.01 | 0.34 | 1.42 |
| Example 2 | 4100 | 8380 | 417 | 75 | ″ | 1.0 | 91.52 | 6.50 | 0.61 | 1.37 |
| | (46.53) | (93.03) | 3.1 | | | 2.0 | 89.46 | 8.56 | 0.29 | 1.69 |
| Example 3 | 4080 | 8340 | 415 | 95 | ″ | 1.0 | 92.89 | 5.13 | 0.59 | 1.39 |
| | (46.30) | (92.58) | 3.1 | | | 2.0 | 88.73 | 9.05 | 0.31 | 1.91 |
| Example 4 | 5000 | 10220 | 258 | 30 | ″ | 1.0 | 90.07 | 8.40 | 0.51 | 1.02 |
| | (56.75) | (113.45) | 1.5 | | | 2.0 | 86.51 | 11.75 | 0.34 | 1.40 |
| Example 5 | | | | 75 | ″ | 1.0 | 91.09 | 7.01 | 0.74 | 1.16 |
| | | | | | | 2.0 | 86.16 | 11.80 | 0.52 | 1.52 |
| Example 6 | | | | 95 | ″ | 1.0 | 90.04 | 8.10 | 0.65 | 1.21 |
| | | | | | | 2.0 | 85.92 | 11.93 | 0.49 | 1.66 |
| Example 7 | 6000 | 12500 | 610 | 30 | ″ | 1.0 | 91.65 | 6.96 | 0.41 | 0.98 |
| | (68.1) | (138.8) | 3.1 | | | 2.0 | 87.63 | 10.79 | 0.27 | 1.31 |
| Example 8 | | | 305 | 30 | ″ | 1.0 | 89.38 | 9.36 | 0.39 | 0.87 |
| | | | 1.5 | | | 2.0 | 86.68 | 11.71 | 0.20 | 1.41 |
| Example 9 | 1000 | 2040 | 103 | 30 | ″ | 1.0 | 89.98 | 8.46 | 0.25 | 1.31 |
| | (11.35) | (22.65) | 3.1 | | | 2.0 | 87.91 | 10.20 | 0.34 | 1.55 |
| | | | | | | 24 | 81.66 | 16.56 | 0.24 | 1.54 |
| Example 10 | 1015 | 2070 | 27 | 30 | ″ | 1.0 | 51.55 | 46.71 | 0.30 | 1.44 |
| | (11.52) | (22.98) | 0.8 | | | 2.0 | 68.18 | 30.10 | 0.26 | 1.46 |
| Example 11 | 1008 | 2060 | 151 | 30 | ″ | 1.0 | 93.07 | 4.18 | 1.12 | 1.63 |
| | (11.44) | (22.87) | 4.5 | | | 2.0 | 90.36 | 6.98 | 0.54 | 2.12 |
| C. Example | 500 | 1020 | — | 30 | ″ | 4.0 | 2.47 | 97.05 | — | 0.10 |
| | (5.67) | (10.29) | | | | 24 | 20.42 | 78.82 | 0.06 | 0.11 |

⁽¹⁾parts by weight of sodium methoxide per 100 parts by weight of 3,4-DHB 100

Referring to Table 1 showing the compositions of the product mixtures in Examples 1 to 6, although the reactions were performed at different temperatures, for example, 30, 75 and 95° C., there was not much difference in production between VEC and a high boiling point material, BMO. Accordingly, it can be noted that the temperature does not much affect the conversion to VEC and the production of the high boiling point material in the VEC synthesis. However, it can be noted that production of VEC is low when the reaction is performed for 2 hours than for 1 hour. In addition, referring to Example 9, when the reaction is performed for 24 hours, the production of VEC is 81.66 wt %, which is lower than when the reaction is performed for 1 hour (89.98 wt %) or 2 hours (87.91 wt %). From the results, it can be concluded that the VEC synthesis reaction is preferably performed for 2 hours or less, and more preferably 1 hour or less. However, for sufficient reaction, the reaction time may be at least 30 minutes.

Referring to Table 1, which shows the compositions of the product mixtures in Examples 7, 8, 10 and 11, when the parts by weight of the base catalyst (sodium methoxide) per 100 parts by weight of 3,4-DHB was at least 1.5 (Examples 7, 8 and 11), the VEC production was at least 86 wt %. However, when the base catalyst was 0.8 parts by weight (Example 10), the VEC production was lower than 70 wt %. In addition, when the parts by weight of the base catalyst was 3.1 or less per 100 parts by weight of 3,4-DHB (Examples 7, 8 and 10), the BMO production was 0.41 wt % or less. When the parts by weight of the base catalyst was 4.5 (Example 11), the BMO production was at least 0.54 wt %. Accordingly, considering the aim of high production of VEC and low production of the high boiling point material (BMO), 1.5 to 3.1 parts by weight of the base catalyst is preferably added per 100 parts by weight of 3,4-DHB.

<Example of Synthesizing VEC 12>

100 g of 3,4-DHB (0.567 mol), 203 g of diethyl carbonate (DEC; 2.26 mol) and 10.3 g of SM-30 (parts by weight of SM per 100 parts by weight of 3,4-DHB: 3.09) were put into a batch-type reactor, and reaction was performed for a total of 4 hours at a temperature of 20° C. and a pressure of 0.2 kgf/cm²G or less.

A composition of a product mixture after 4-hour reaction was 9.7 wt % VEC, 29.6 wt % unreacted 3,4-DHB, 44.6 wt % EtOH, 5.6 wt % of a first unknown material, 9.2 wt % of a second unknown material, 0.51 wt % BMO and 0.79 wt % of another high boiling point material. This composition is significantly different from when DMC was used.

<Example of Purifying VEC>

The product mixture obtained under the same reaction conditions as Example 1 was put into a reboiler of a solvent removing distiller, which was operated at 60° C. to remove methanol. Afterward, the resulting product was heated at 90° C. to remove unreacted dimethyl carbonate (DMC).

10.8 kg of the mixture from which methanol and DMC were removed was put into a reboiler of a first purification distiller. The first purification distiller was operated at a reboiler temperature of 105° C. and a distillation column pressure of 1.0 torr or less to remove methanol, unreacted DMC, unreacted 3,4-DHB and moisture remaining in the mixture. The total amount of the removed materials was 2.26 kg (containing 34.7 wt % VEC). At this time, the remaining mixture in the reboiler of the first purification distiller contained about 99.1 wt % VEC, about 3.0 wt % 3,4-DHB, about 0.6 wt % of other materials and a miscellaneous sticky material. 40 ppm moisture was also contained.

After that, the first purification distiller was continuously operated for 20 hours at a reboiler temperature of 115° C., a distillation column pressure of 0.5 torr or less and a condenser temperature of 30° C., thereby obtaining 8.04 kg of a first purified product, crude VEC.

8.04 kg of the first purified product, crude VEC, was put into a reboiler of a second purification distiller, which was operated at a reboiler temperature of 110° C., a distillation column pressure of 1.0 torr or less and a condenser temperature of 30° C. to remove unreacted 3,4-DHB and impurities from a top portion of the distillation column. In this process, a refluxed solution was obtained from a side-curt in the middle of the distillation column to analyze. When the purity of VEC in the refluxed solution was 99.9 wt %, the reboiler temperature of the second purification distiller was increased to 115° C. and operated, thereby obtaining 6935 g of a final purified product, VEC product. Here, the final product was composed of 99.93 wt % VEC and 5 ppm moisture, which contained a very low level of moisture and high-purity VEC. The yield of VEC was 89.64% and the distillation efficiency was 86.2%. Here, the remaining mixture in the reboiler was composed of 99.44 wt % VEC, 0.09 wt % 3,4-DHB and 0.47 wt % of other components.

According to the present invention, VEC may be manufactured by a single process performed in a reactor. To be specific, VEC may be synthesized without an additional process by reaction of 3,4-DHB and dialkyl carbonate in the presence of a base catalyst in the reactor. Also, the product mixture containing VEC may be simply separated and purified by distillation at sub-atmospheric pressure, thereby obtaining high-purity VEC. The method according to the present invention is simpler, safer and more economical than other methods of manufacturing VEC, and it yields high-purity VEC.

Although a few embodiments of the present invention have been shown and described, it would be appreciated by those skilled in the art that changes may be made in this embodiment without departing from the principles and spirit of the invention, the scope of which is defined by the claims and their equivalents.

What is claimed is:

1. A method of manufacturing vinylethylene carbonate, comprising:
synthesizing vinylethylene carbonate by reaction of 3,4-dihydroxy-1-butene and dialkyl carbonate using a base catalyst.

2. The method according to claim 1, wherein the dialkyl carbonate includes dimethyl carbonate.

3. The method according to claim 1, wherein the base catalyst includes sodium methoxide.

4. The method according to claim 1, wherein the reaction is performed at a temperature of 30 to 100° C. and a pressure of 0 to 0.2 kgf/cm²G.

5. The method according to claim 1, wherein the dialkyl carbonate is added in an amount of 1.5 to 2.5 moles per 1 mole of 3,4-dihydroxy-1-butene.

6. The method according to claim 1, wherein the base catalyst is added in an amount of 1.5 to 3.1 parts by weight per 100 parts by weight of 3,4-dihydroxy-1-butene.

7. A method of manufacturing vinylethylene carbonate, comprising:
putting 3,4-dihydroxy-1-butene, dialkyl carbonate and a base catalyst into a reactor;
obtaining a product mixture containing vinylethylene carbonate and alcohol by reaction of 3,4-dihydroxy-1-butene and dialkyl carbonate in the reactor;
removing the alcohol from the obtained product mixture; and
distilling the alcohol-free mixture to obtain purified vinylethylene carbonate.

8. The method according to claim 7, wherein the distilling the alcohol-free mixture is performed by putting the alcohol-free mixture into a distiller and operating the distiller at sub-atmospheric pressure.

9. The method according to claim 8, wherein the distilling the alcohol-free mixture includes obtaining a first purified product by first distillation of the alcohol-free mixture in a first distiller, and obtaining a second purified product by second distillation of the first purified product in a second distiller.

10. The method according to claim 9, further comprising:
operating the first distiller at a lower temperature than for the first distillation to remove an unreacted reactant, alcohol and moisture, before the first distillation of the alcohol-free mixture; and
operating the second distiller at a lower temperature than for the second distillation to remove an unreacted reactant, alcohol and moisture, before the second distillation of the first purified product.

11. The method according to claim 9, further comprising:
removing moisture from the first purified product before the first purified product is put into the second distiller.
